SEQUENCE LISTING (24 sequences)

SEQ ID NO: 1 - protein, 15 aa, HORDEUM VULGARE CV. SCARLETT
  misc_feature: N-terminal end of soluble NPPase
AAVRASPDLLGSRGE

SEQ ID NO: 2 - protein, 11 aa, HORDEUM VULGARE CV. SCARLETT
  misc_feature: Tryptic sequence of soluble NPPase
ASYPGXTSXXR

SEQ ID NO: 3 - protein, 11 aa, HORDEUM VULGARE CV. SCARLETT
  misc_feature: Tryptic sequence of soluble NPPase
HAPADTVTXGR

SEQ ID NO: 4 - protein, 5 aa, HORDEUM VULGARE CV. SCARLETT
  misc_feature: Tryptic sequence of soluble NPPase
APPYP

SEQ ID NO: 5 - protein, 8 aa, HORDEUM VULGARE CV. SCARLETT
  misc_feature: Tryptic sequence of soluble NPPase
AWVTVEFK

SEQ ID NO: 6 - protein, 8 aa, HORDEUM VULGARE CV. SCARLETT
  MISC_FEATURE: Tryptic sequence of soluble NPPase
XSXEGXWR

SEQ ID NO: 7 - protein, 15 aa, ORYZA SATIVA
  misc_feature: N-terminal end of soluble NPPase
GSAFVSATPALLGDQ

SEQ ID NO: 8 - protein, 23 aa, ORYZA SATIVA
  misc_feature: Tryptic sequence (MS/MS) of soluble NPPase
FQLLNQRYDFSFALETGGLENPK

SEQ ID NO: 9 - protein, 11 aa, ORYZA SATIVA
  misc_feature: Tryptic sequence (MS/MS) of soluble NPPase
LVAVSEALSFK

SEQ ID NO: 10 - protein, 10 aa, ORYZA SATIVA
  misc_feature: Tryptic sequence (Edman) of soluble NPPase
LAQGKSYDEM

SEQ ID NO: 11 - protein, 10 aa, ORYZA SATIVA
  misc_feature: Tryptic sequence (MS/MS) of soluble NPPase
TAAGTLTFNR

SEQ ID NO: 12 - protein, 11 aa, ORYZA SATIVA
  misc_feature: Tryptic sequence (MS/MS) of soluble NPPase
DPGFLHTAFLR

SEQ ID NO: 13 - protein, 12 aa, ORYZA SATIVA
  misc_feature: Tryptic sequence (MS/MS) of soluble NPPase
APDFPGQNSLQR

SEQ ID NO: 14 - protein, 9 aa, ORYZA SATIVA
  misc_feature: Tryptic sequence (MS/MS) of soluble NPPase
IIVFGDMGK

SEQ ID NO: 15 - protein, 12 aa, ORYZA SATIVA
  misc_feature: Tryptic sequence (MS/MS) of soluble NPPase
DWPNTGGFFDVK

SEQ ID NO: 16 - protein, 11 aa, ORYZA SATIVA
  misc_feature: Tryptic sequence (MS/MS) of soluble NPPase
FIEQCLSTVDR

SEQ ID NO: 17 - protein, 9 aa, ORYZA SATIVA
  misc_feature: Tryptic sequence (MS/MS) of soluble NPPase
VYDSFYVER

SEQ ID NO: 18 - DNA, 18 bp, ORYZA SATIVA
  misc_feature: Primer of the 5′ region of NPPase
ggcgttgctcggcgacca

SEQ ID NO: 19 - DNA, 19 bp, ORYZA SATIVA
  misc_feature: Primer of the 3′ region of NPPase
gaggcgagcgtggtgggga

SEQ ID NO: 20 - DNA, 2186 bp, ORYZA SATIVA
  misc_feature: complete cDNA of rice NPPase
tttgaattagctagctgtagaaagctagccatggttagtaggaagagaggaggaggaggaggcgtggcaatggcggtggcgatgctgctggcggcggcgagcgcgtcgcggccgtcgtcgtcgctggaagggttccagccgctgtcgaagatcgccgtccacaaggccaccgtcgacctccacggctccgcgttcgtcagcgccacgccggcgttgctcggcgaccagggagaagacacagagtgggtcacggtgaaatacggctgggcaaacccttccgctgacgactggattgctgtcttctctccggccgatttcatctcgggttcttgccctaatccttccagatacccggatgagccgctgctctgcactgcaccaataaagtatcaattcgcaaactactcggcgaactacgtgtactggggcaagggcagcatccggttccagctcatcaaccagcgctacgacttctccttcgccctgttcaccggcggcctggaaaaccctaagctggtggcggtgtcggaggcgatatcgttcaagaaccccaaggcgccggtgtaccctcggctggcgcagggcaagtcgtacgacgagatgaccgtcacatggaccagcggctacgacatcagcgaggcgtacccgttcgtcgagtggggcatggtcgtcgccggcgccgccgctccaacccgcaccgccgccggcacgctcaccttcaaccgcggcagcatgtgcggtgacccggaccgcactgttgggtggagagaccccgggttcatccacacagctttcctgagagacctgtggcccaacaaagagtactactacaagattgggcatgaactttctgacggatcaattgtctggggcaagcaatacactttccgggcgccacccttccctggccagaactcgctgcaacgcatcatcgtcttcggcgacatgggcaaggcggagagagacggatcaaacgagttcgccaactaccagccaggatctctgaacacgacggacaggctggtcgaggatctggacaactacgacattgtcttccacatcggtgatcttccgtacgccaatggctacatctcccagtgggaccagttcaccgcccaggtcgcccccatcaccgccaagaagccctacatgattgcaagcggtaaccatgagagggactggcccaacaccggagggttcttcgacgtcaaggactccggcggcgagtgcggcgttccggcagagaccatgtactactacccggccgagaatcgagccaacttctggtacaaggtggactacgggatgttccggttctgcatcgcggactcggagcacgactggagggagggtaccgaccagtacaagttcatcgagcagtgcctgtcgacggtggaccggaagcaccagccgtggctcatcttcgcggcgcaccgcgtgctgggctactcctccaactggtggtacgccgaccagggctccttcgaggagcccgaagggagggagagcctgcagcggctgtggcagcgccaccgcgtcgacgtcgccttcttcggccacgtccacaactacgagcggacgtgcccgatgtaccagagccagtgcgtctccggcgagaggcgccgctactccggcaccatgaacggcaccatcttcgtcgtcgccggcggcggcgggagccacctctcggactacacctcggcgatccccaagtggagcgttttcagggaccgggacttcgggttcgtcaagctcaccgcgttcaaccactcgtcgctgctgttcgagtacaagaagagcagcgatgggaaggtgtatgactccttcaccgtggagagggattaccgcgacgtgctcagctgcgtgcacgacagctgcctccccaccacgctcgcctcctgatgaatgaaacaagggaaaggatcattattaggatgcatgagttgatgcctcatcgtcaaaatgctggcagctgagaaagagtgatcggtcggtcgatcgagttgggttttattttttttcttcttcttcaaccatttcgatcaggtgtggtagtggtcgatcgcttggctcgatcgtgtttctcttcctcatggatggtgatgttgtgcaataaaattgcttagctgctcgggcacaaatgtctaaaaaaaaaaaaaaaaaaaaaaaaaaaaaaaaaaaaaaaa

SEQ ID NO: 21 - protein, 623 aa, ORYZA SATIVA
  misc_feature: Amino acid sequence of rice NPPase
MVSRKRGGGGGVAMAVAMLLAAASASRPSSSLEGFQPLSKIAVHKATVDLHGSAFVSATPALLGDQGEDTEWVTVKYGWANPSADDWIAVFSPADFISGSCPNPSRYPDEPLLCTAPIKYQFANYSANYVYWGKGSIRFQLINQRYDFSFALFTGGLENPKLVAVSEAISFKNPKAPVYPRLAQGKSYDEMTVTWTSGYDISEAYPFVEWGMVVAGAAAPTRTAAGTLTFNRGSMCGDPDRTVGWRDPGFIHTAFLRDLWPNKEYYYKIGHELSDGSIVWGKQYTFRAPPFPGQNSLQRIIVFGDMGKAERDGSNEFANYQPGSLNTTDRLVEDLDNYDIVFHIGDLPYANGYISQWDQFTAQVAPITAKKPYMIASGNHERDWPNTGGFFDVKDSGGECGVPAETMYYYPAENRANFWYKVDYGMFRFCIADSEHDWREGTDQYKFIEQCLSTVDRKHQPWLIFAAHRVLGYSSNWWYADQQSFEEPEGRESLQRLWQRHRVDVAFFGHVHNYERTCPMYQSQCVSGERRRYSGTMNGTIFVVAGGGGSHLSDYTSAIPKWSVFRDRDFGFVKLTAFNHSSLLFEYKKSSDGKVYDSFTVERDYRDVLSCVHDSCLPTTLAS

SEQ ID NO: 22 - DNA, 1268 bp, HORDEUM VULGARE CV. SCARLETT
  misc_feature: Incomplete cDNA of barley NPPase
ctccgacggaagcgtggtgtgggccaagccctacactttccgggcaccgccaacccccgggcagaactcgctgcagcgtatcatcgtcttcggtgacatgggaaaggcggagagggacggatcaaacgagttcgccaactaccagccggggtcgctcaacacgacggacaggctgattgaagatctggacaactacgacatcgtcttccacatcggcgacatgccctacgccaacgggtacctctcccagtgggaccagttcaccgcacaggtcgcccccatcagcgccaagaaaccctacatggttgcaagcggcaaccacgagagggactggcccaacaccggcgggttcttcgacgtcaaggactccggcggcgaatgcggcgtgccggccgagaccatgtactactaccccgccgaaaacagggcaaacttctggtacaaggtggactacgggatgttccggttctgcgtgggggactcggagcacgactggagggagggcaccccgcagtacaagttcatcgaggagtgcctgtcgacggtggaccggaagcaccagccgtggctcatcttcacggcgcaccgggtgctgggctactcctccaactcgtggtacgccgaccagggctccttcgaggagcccgagggacgggagagcctgcagaagctgtggcagcgctaccgcgtcgacatcgcctccttcggccacgtccacaactacgagcgcacatgcccgctctaccagagccagtgcgtcaacgccgacaagacccactactcgggcaccatgaacggcaccatcttcgtcgtcgccggcgggggcggcagccacctgtcgtcctacaccaccgccatccccaagtggagcatattcagggaccatgactacgggttcaccaagctcaccgcattcaaccactcctcgcttctcttcgagtacatgaagagcagcgacggcaaggtctacgactccttcaccatccacagggattaccgcgacgtgctcagctgcgtgcacgacagctgcttccccaccacgctcgctagctagctcatatcgtccggccgtcatgtcaatgtaatggagggtcatccatccaataaaattgtgggcatgtgttgagtaataaaattggtcagctgcacaatttatatgtgctagtaaaaagatcatgcaagaggtgggtgtatgctcgttatatatgctttgtaactccttcatgtcatattattatgggttaataaaaacatcctttattaaaaaa

SEQ ID NO: 23 - protein, 350 aa, HORDEUM VULGARE CV. SCARLETT
  misc_feature: Amino acid sequence deduced from the cDNA of barley NPPase
SDGSVVWAKPYTFRAPPTPGQNSLQRIIVFGDMGKAERDGSNEFANYQPGSLNTTDRLIEDLDNYDIVFHIGDMPYANGYLSQWDQFTAQVAPISAKKPYMVASGNHERDWPNTGGFFDVKDSGGECGVPAETMYYYPAENRANFWYKVDYGMFRFCVGDSEHDWREGTPQYKFIEECLSTVDRKHQPWLIFTAHRVLGYSSNSWYADQGSFEEPEGRESLQKLWQRYRVDIASFGHVHNYERTCPLYQSQCVNADKTHYSGTMNGTIFVVAGGGGSHLSSYTTAIPKWSIFRDHDYGFTKLTAFNHSSLLFEYMKSSDGKVYDSFTIHRDYRDVLSCVHDSCFPTTLAS

SEQ ID NO: 24 - DNA, 39 bp, HORDEUM VULGARE CV. SCARLETT
  misc_feature: Primer of the 5′ region of NPPase
gcagcagtacgagcatcaccagatctactagcatcacga

(19) United States
(12) Patent Application Publication
(10) Pub. No.: US 2006/0242739 A1
(43) Pub. Date: Oct. 26, 2006

(54) Plant nucleotide-sugar pyrophosphatase/phosphodiesterase (nppase), method of obtaining same and use of same in the production of assay devices and in the production of transgenic plants

(75) Inventor: Francisco Jose Munoz Perez, Pamplona-Navarro (ES)

Correspondence Address: LADAS & PARRY, 26 WEST 61ST STREET, NEW YORK, NY, 10023, US

(73) Assignees: Universidad Publica De Navarra, Navarra (ES); Niigata University, Niigata City, Niigata Prefecture (JP)

(21) Appl. No.: 10/520,696

(22) PCT Filed: Jul. 15, 2003
(86) PCT No.: PCT/ES03/00363

§ 371 Date: Oct. 19, 2005

(30) Foreign Application Priority Data
Jul. 15, 2002 (ES) P200201647
Nov. 20, 2002 (ES) P200202673

(51) Int. Cl. A01H 5/00; C12Q 1/68; C07H 21/04; C12N 9/00; C12N 15/82; C12N 5/04

(52) U.S. Cl. 800/294; 435/6; 435/183; 435/419; 435/468; 536/23.2

(57) ABSTRACT

Plant nucleotide pyrophosphatase/phosphodiesterase (NPPase), method of production, use in the manufacture of testing devices and in the production of transgenic plants. NPPase is an enzyme that catalyses the hydrolysis of a wide range of small molecules with phosphodiester and phosphosulphate bonds, in particular ADPG (adenosine diphosphate glucose) and APS (adenosine 5′-phosphosulphate). The enzyme obtained from plant extracts is used in assay devices for determining levels of nucleoside diphosphate sugars, based either on the sugar-1-phosphate released, or on the nucleoside monophosphate, both of which are products formed by the reaction catalysed by NPPase, as well as the detection of sulphonucleotides such as 3′-phosphoadenosine 5′-phosphosulphate (PAPS) and APS. The amino acid sequence of the enzyme is also described, as well as the nucleotide sequence of a complete cDNA and another incomplete cDNA. Finally, it describes the production of transgenic plants that overexpress NPPase and that have a high content of sugars, low content of starch and cell-wall polysaccharides and high resistance to high concentrations of salts and high temperature.

### INDUSTRIAL FIELD TO WHICH THE INVENTION RELATES

The invention relates to the field of the production, purification and characterization of various isoforms of nucleotide pyrophosphatase/phosphodiesterase (NPPase) especially of rice and barley, and to the applications of this enzyme in the determination of levels of nucleotide sugars, and of sulphonucleotides, as well as in the production of transgenic plants in which there is overexpression of cDNA's of the genes that code for the said isoforms of NPPase, giving rise to plants with reduced content of starch and cell-wall polysaccharides and high resistance to salinity and temperature.

### STATE OF THE PRIOR ART

Starch is the principal storage form of carbohydrates in plants. It is accumulated in large quantities in organs such as seeds (wheat, barley, maize, pea, etc.) and tubers (potato and yam among others), and is a fundamental constituent of the human diet. On the other hand, starch is a polymer that is often used in the paper, cosmetic, pharmaceutical and food industries, and is also used as a basic component for the manufacture of biodegradable plastics and environment-friendly paints. Another polysaccharide, cellulose, is a fundamental component of the cell wall of plants, constituting the fundamental raw material in such important processes as papermaking. Consequently, investigation of the processes involved in the synthesis of these polymers of glucose is given priority in various areas of industrial production. UDPglucose (UDPG) is the fundamental precursor of the biosynthesis of cellulose and of cell-wall polysaccharides. It is also the precursor molecule of processes connected with the glycosylation of proteins and lipids. On the other hand, ADPglucose (ADPG) is the universal precursor of the biosynthesis of starch in storage tissues of plants. Its concentration in the cell is determining for the quantity and quality of the starch produced by the plant. Considerations of the factors that govern the endogenous levels of ADPG and UDPG in the plant cell have basically revolved around their synthesizing enzymes, such as ADPG pyrophosphorylase, UDPG pyrophosphorylase and sucrose synthase (Preiss, (1988) “Biosynthesis of starch and its regulation”. The Biochemistry of Plants. Vol. 14, Academic Press, New York, pp. 182-249; Pozueta-Romero, J., Perata, P., Akazawa, T. (1999) “Sucrose-starch conversion in heterotrophic tissues”. Crit. Rev. Plant Sci. 18, 489-525). However, there has been little study of the mechanisms responsible for the degradation of these nucleotide sugars (Feingold, D. S., Avigad, G. (1980) “Sugar transformation in plants”. The Biochemistry of Plants. Vol. 3, Stumpf, P. K. and Conn, E. E. eds. Academic Press, New York, pp. 101-170). There are indications that both bacteria and mammals possess enzymatic mechanisms capable of hydrolysing ADPG and UDPG (Melo, A., Glaser, L. (1966) “Nucleotide diphosphate hexose pyrophosphatases”. Biochem. Biophys. Res. Commun. 22, 524-531; Bessman, M. J., Frick, D. N., O'Handley, S. F. (1996) “The MutT proteins or Nudix hydrolases, a family of versatile, widely distributed housecleaning enzymes”. J. Biol. Chem. 271, 25059-25062; Rodríguez, P., Bass, S. T., Hansen, R. G. (1968) “A pyrophosphatase from mammalian tissues specific for derivates of ADP”. Biochim. Biophys. Acta, 167, 199-201; Gasmi, L., Cartwright, J. L., McLennan, A. G. (1999) “Cloning, expression and characterization of YSA1H, a human adenosine 5′-diphosphosugar pyrophosphatase possessing a MutT motif”. Biochem. J. 331-337; Moreno-Bruna, B., Baroja-Fernández, E., Muñoz, F. J., Bastarrica-Berasategui, A., Zandueta-Criado, A., Rodríguez-López, M., Akazawa, T., Pozueta-Romero, J. (2001) “Adenosine diphosphate sugar pyrophosphatase prevents glycogen biosynthesis in Escherichia coli”. Proc. Natl. Acad. Sci. 98, 8128-8132). In plants, activity of this kind has barely been described in the literature (Salvucci, M. E., Crafts-Brandner, S. J. (1995) “Purification and properties of a unique nucleotide pyrophosphatase/phosphodiesterase I that accumulates in soybean leaves in response to fruit removal”. Plant Physiol. 108, 1269-1276; Rodríguez-López, M., Baroja-Fernández, E., Zandueta-Criado, A., Pozueta-Romero, J. (2000) “Adenosine diphosphate glucose pyrophosphatase: a plastidial phosphodiesterase that prevents starch biosynthesis”. Proc. Natl. Acad. Sci., 97, 8705-8710; Baroja-Fernández, E., Zandueta-Criado, A., Rodríguez-López, M., Akazawa, T., Pozueta-Romero, J. (2000) “Distinct isoforms of ADPglucose pyrophosphatase and ADPglucose pyrophosphorylase occur in the suspension-cultured cells of sycamore (Acer pseudoplatanus L.). FEBS Lett. 480, 277-282; Rodríguez-López, M., Baroja-Fernández, E., Zandueta-Criado, A. Moreno-Bruna, B. Muñoz, F. J., Akazawa, T., Pozueta-Romero, J. (2001) “Two isoforms of a nucleotide-sugar pyrophosphatase/phosphodiesterase from barley leaves (Hordeum vulgare L.) are distinct oligomers of HvGLP1, a germin-like protein”. FEBS Lett. 490, 44-48).

In various industries, starch is a basic essential as a viscosity-increasing and gelling agent. The biosynthesis of starch in the plant cell starting from ADPG takes place in the subcellular compartment called the plastid. ADPG is both synthesized and utilized in this compartment, and therefore the levels of starch can be controlled by controlling the processes that regulate the ADPG levels. The various applications of starch produced in a plant are based on the balance of amylose and amylopectin, which determines the structure of the starch granule, as well as its viscosity in aqueous suspensions. The ratio of amylose to amylopectin depends on the concentration of ADPG in the plant cell. To date, no method is known for regulating the characteristics of the starch produced in a plant by controlling the degradation of ADPG, which the enzyme described in the present invention, can provide. In addition to acting as a storage substance for the plant, starch accumulates in the plant cell in circumstances where the plant is not subject to conditions of water stress. In conditions where the plant is subjected to high temperatures or high concentrations of salts in the environment, the plant stops accumulating starch, and produces large quantities of soluble sugars that accumulate in the vacuole (Keeling, P. L., Bacon, P. J., Holt, D. C. (1993) “Elevated temperature reduces starch deposition in wheat endosperm by reducing the activity of soluble starch synthase” Planta 191, 342-348; Geigenberger, P., Geiger, M., Stitt, M. (1998) “High-temperature perturbation of starch synthesis is attributable to inhibition of ADP-glucose pyrophosphorylase by decreased levels of glycerate-3-phosphate in growing potato tubers” Plant Physiol. 117, 1307-1316). In addition to these adaptive changes of carbohydrate metabolism to water stress, the plant undergoes changes in its sulphur metabolism, avoiding the accumulation of adenosine-5′-phosphate (PAP) arising from the transformation of adenosine 5′-phosphosulphate (APS) and 3′-phosphoadenosine 5′-phosphosulphate (PAPS) (Gil-Mascarell, R., López-Coronado, J. M., Bellés, J. M., Serrano, R., Rodríguez, P. L. (1999) “The Arabidopsis HAL2-like gene family includes a novel sodium-sensitive phosphatase” Plant J. 17, 373-383). On the basis of these observations, it is possible that enzymatic reactions responsible for the hydrolysis of ADPG, APS and PAPS are responsible for adaptive processes of the plant to the conditions of water stress.

Chromatographic and radiological techniques are powerful tools for determining levels of nucleotides such as sulphonucleotides (APS and PAPS among others; Yoshida, H., Fukui, S., Yamashina, I., Tanaka, T., Sakano, T., Usui, T., Shimotsuji, T., Yabuuchi, H., Owada, M., Kitagawa, T. (1982) “Elevation of nucleotide pyrophosphatase activity in skin fibroblasts from patients with Lowe's syndrome”. Biochem. Biophys. Res. Commun. 107, 1144-1150) and nucleoside diphosphate sugars (such as the derivatives of glucose, ribose, mannose, galactose, glucuronic acid, fructose and galacturonic acid) in raw extracts of animal, plant or microbial origin. Although of very general use, they require a considerable investment in equipment and in the preparation of the test samples. Unfortunately, apart from a few exceptions (Puhakainen, E., Saarinen, A., Hänninen, 0. (1977) “UDPglucuronic acid pyrophosphatase assay with the aid of alkaline phosphatase” Acta Chem. Scandinavica B31, 125-129) scant use is made of possible alternative methods that permit the detection and quantification of nucleotide sugars and sulphonucleotides in a simple and efficient manner. Analysis of the blood, muscle, kidney and liver levels of some of the aforementioned nucleotide sugars is important in clinical practice (Cortes, P., Dumler, F., Sastry, K. S., Verghese, C. P., Levin, N. W. (1982) “Effects of early diabetes on uridine diphosphosugar synthesis in the rat renal cortex”. Kidney Int. 21, 676-682; Spiro, M. J. (1984) “Effect of diabetes on the sugar nucleotides in several tissues of the rat” Diabetologia 26, 70-75; Sochor, M., Kunjara, S., Baquer, N. Z., McLean, P. (1991) “Regulation of glucose metabolism in livers and kidneys of NOD mice”. Diabetes 40, 1467-1471). For example, since UDPG is the precursor of glycogen in animals, analysis of the levels of this molecule can be important in the investigation and diagnosis of diseases associated with carbohydrate metabolism, such as various types of diabetes. On the other hand, determination of the levels of PAPS in the urine is essential for the diagnosis of serious illnesses such as Lowe's syndrome or antiphospholipid syndrome (Yoshida, H., Fukui, S., Yamashina, I., Tanaka, T., Sakano, T., Usui, T., Shimotsuji, T., Yabuuchi, H., Owada, M., Kitagawa, T. (1982) “Elevation of nucleotide pyrophosphatase activity in skin fibroblasts from patients with Lowe's syndrome”. Biochem. Biophys. Res. Commun. 107, 1144-1150; Amigo, M. C., García-Torres, T. (2000) “Morphology of vascular, renal, and heart lesions in the antiphospholipid syndrome: relationship to pathogenesis” Curr. Rheumatol. Rep. 2000, 2, 262-270). Obviously the possibility of analysing, simply and inexpensively, the levels of these substances in a sample represents an advantageous alternative to the chromatographic techniques.

The invention describes the purification and applications of an enzymatic product of plant origin that we designate NPPase, which catalyses the hydrolysis of small molecules with phosphodiester or phosphosulphate bonds and in particular ADPG and APS, as the preferred substrates. In the first priority (ES 200201647), with the experimental data available to them at that time, the inventors tentatively designated the enzymatic product isolated as NSPPase, but in the later, second priority (ES 200202673), with the data that had been accumulated, its designation was changed to NPPase as at present.

The plant enzyme of the invention has various isoforms in the plant tissues from which it can be obtained (Baroja-Fernández, E., Zandueta-Criado, A., Rodríguez-López, M., Akazawa, T., Pozueta-Romero, J. (2000) “Distinct isoforms of ADPglucose pyrophosphatase and ADPglucose pyrophosphorylase occur in the suspension-cultured cells of sycamore (Acer pseudoplatanus L.). FEBS Lett. 480, 277-282). The isoform that is simplest to extract is the one we call soluble, whereas other isoforms, which we can call particulates, are found firmly adhering to the starch granules.

In the present invention we succeeded in purifying and partially sequencing various soluble isoforms of NPPase of barley and rice with approximate sizes of 70 kDa. On the basis of these sequences, we were able to isolate the cDNA's that code for the NPPases. After comparing their sequences with those available in databases, it was observed that the NPPases of rice and barley share homology with PPD1, a nucleotide phosphatase/phosphodiesterase of Lupinus luteus which, in contrast to the NPPase of this invention, has as its best substrates the di- and tri-phosphate nucleosides but does not hydrolyse nucleotide sugars (Olczak, M., Olczak, T. (2002) “Diphosphonucleotide phosphatase/phosphodiesterase from yellow lupin (Lupinus luteus L.) belongs to a novel group of specific metallophosphatases”. FEBS Lett. 519, 159-163). Moreover, it shares homology with other sequences that code for unknown or possible proteins of dicotyledonous plants such as Arabidopsis and chickpea. One object of the invention is, firstly, the production, characterization and sequencing of various soluble 70-kDa isoforms of NPPase in substantially pure form, starting from plant tissues of barley (Hordeum vulgare) and rice (Oryza sativa). Another object is the production of complete cDNA's that code for the NPPases and contrasting them with sequences available in databases. The design of constructions derived from the cDNA's of the soluble NPPases intended for the production of transgenic plants with high NPPase activity whose content and quality of the starch, as well as that of cell-wall polysaccharides, are modified relative to control plants, is detailed later. The said plants do not accumulate the marker of osmotic toxicity PAP, so that they are more resistant to high concentrations of salts than the control plants.

Another object of the invention is the method used for making devices or kits for determination of nucleoside diphosphate sugars and sulphonucleotides based on the use of the enzymatic product with NPPase activity.

### DETAILED DESCRIPTION OF THE INVENTION

The plant product with NPPase enzyme activity according to the invention can be obtained and purified starting from any plant tissue from any species, i.e. any monocotyledon or dicotyledon, for example barley (Hordeum vulgare), wheat (Triticum aestivum), rice (Oryza sativa), pepper (Capsicum annuum), tomato (Lycopersicon sculentum), potato (Solanum tuberosum), Arabidopsis (Arabidopsis thaliana), or sycamore (Acer pseudoplatanus L.), to cite just some of the numerous representative examples of different families and genera.

Production and Purification of a Soluble Isoform of NPPase:

The general method for obtaining and purifying the soluble plant NPPase described in the invention includes the following steps, in which slight variations are admissible that do not substantially alter the general scheme of the method of extraction and purification. NPPase is especially abundant in young leaves and is practically absent from storage tissues such as endosperms of seeds and tubers, accordingly it is recommended to use young leaves for the extraction of NPPase.

1. Homogenization of the plant tissue with an extraction buffer.

2. Filtration through four layers of Miracloth® (filter cloth for milk whey used in cheesemaking).

3. Ultracentrifugation of the filtered homogenate.

4. Precipitation of the proteins of the supernatant in ammonium sulphate.

5. Resuspension of the precipitate in buffer with pH of 4.2.

6. Heating for at least 15 minutes at a temperature between 60 and 65° C.

7. Centrifugation.

8. Concentration of the supernatant and purification of the protein by gel-filtration chromatography. The enzyme activity of NPPase is detected by detecting the production of G1P and AMP in samples incubated with substrates such as UDPG or ADPG.

9. Application of the NPPase to affinity column chromatography of the Concanavalin A type, which indicates that the NPPase is glycosylated.

10. Isoelectric focusing in a Multiphor II system, using PAGplates with pH range between 3.5 and 9.5 (Amersham/Pharmacia). The position of the NPPase can be determined easily in one of the following ways:

a) Elution of the protein followed by detection of the production of G1P in the presence of ADPG or UDPG.

b) Incubation of the gel in a solution with bis-paranitrophenylphosphate (bis-PNPP) and development in a basic solution as described by Nishimura and Beevers (Nishimura, M., Beevers, H. (1978). Plant Physiol. 62, 44-48).

11. Separation of the protein in denaturing gel by electrophoresis in a system of neutral or slightly acid buffers such as NuPAGE 4-12% Bis-Tris (Novex, San Diego, Calif.). The position of the NPPase can be determined easily in one of the following ways:

a) Elution of the protein followed by detection of the production of G1P in the presence of ADPG.

b) Incubation of the gel in a solution with bis-PNPP and development in a basic solution.

Identification of the Product with NPPase Enzyme Activity

The enzyme product obtained by the methods described above, or other equivalent methods, is identified by means of the following functional standards:
It is a pyrophosphatase/phosphodiesterase that catalyses the hydrolysis of ADPG in equimolar quantities of G1P and AMP (Rodríguez-López, M., Baroja-Fernández, E., Zandueta-Criado, A., Pozueta-Romero, J. (2000) “Adenosine diphosphate glucose pyrophosphatase: a plastidial phosphodiesterase that prevents starch biosynthesis”. Proc. Natl. Acad. Sci., 97, 8705-8710)
In addition to ADPG, it recognizes small molecules that possess phosphodiester and phosphosulphate bonds, such as UDPG, GDP-glucose, GDP-mannose, ADP-mannose, bis-PNPP, PAPS and APS and others of similar structure.
It does not hydrolyse molecules with phosphomonoester bonds such as G1P, G6P, AMP, 3-phosphoglycerate, and other similar ones. Nor does it hydrolyse cyclic AMP or long-chain nucleic acids such as DNA or RNA, which are substrates of other phosphodiesterases described in the literature.
In contrast to pyrophosphatases of ADP-sugars (EC 3.6.1.13, EC 3.6.1.21) described in bacteria and animals and in contrast to other phosphodiesterases (EC 3.1.4), its ionic requirements are reduced, therefore it can work in the absence of ions of magnesium, manganese, cobalt, and other divalent cations.
In contrast to the pyrophosphatases of nucleoside diphosphate sugars of bacteria and animals, NPPase hydrolyses bis-PNPP.
It is inhibited by phosphorylated molecules such as AMP, ADP, ATP, 3-phosphoglycerate, orthophosphate, inorganic pyrophosphate, and others with similar characteristics.
It is strongly inhibited by molybdate and arsenate.
It is resistant to ionic detergents such as SDS (sodium dodecylsulphate).
It is resistant to the action of a wide range of proteases, such as Proteinase K and Pronase (Boehringer).
Its activity is not affected by the action of typical inhibitors of phosphodiesterases such as β-mercaptoethanol, EDTA, reduced cysteine, ascorbate, and other reducing and chelating agents.
It is sensitive to slightly basic pH and is very stable at pH between 4 and 7.5.

Production of a Complete cDNA that Codes for the Soluble NPPase

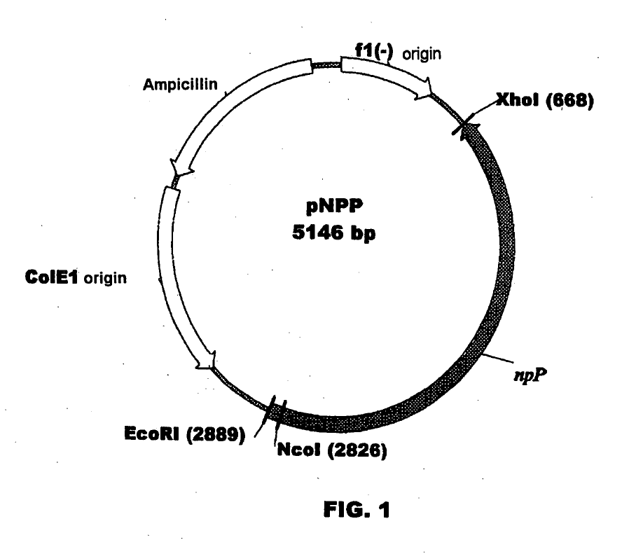

Some known internal amino acid sequences of the various NPPases purified were compared with others present in the databases. The result of this analysis made it possible to design two primers that were used for obtaining, by RT-PCR, a cDNA that codes for an NPPase of rice and of barley. The cDNA's obtained were cloned in the pGEM-T vector and were used as probes for searching for a complete cDNA in the cDNA library of young rice leaves. The complete cDNA obtained was introduced in the EcoRV restriction site of the plasmid pSK Bluescript (Stratagene) giving rise to the construction pNPP (FIG. 1) which was amplified in the host bacterium XL1 Blue. A strain of this bacterium was deposited on 15 Oct. 2002 in the Spanish Type Culture Collection, University of Valencia, Research Building, Campus of Burjasot, 46100 Burjasot, Valencia, with the number CECT 5739.

Production of Transgenic Plants that Overexpress the cDNA of Soluble NPPase

pNPP was digested successively with the enzymes HindIII, T4 DNA polymerase and XbaI. The fragment released (which contains the cDNA of NPPase) was cloned in the plasmid pVT′BSP after being digested successively by the enzymes NcoI, T4 DNA polymerase and XbaI. In this way we obtain a plasmid designated p35SNPPNOS which has the constitutive promoter 35S, the cDNA of NPPase and the Nos terminator.

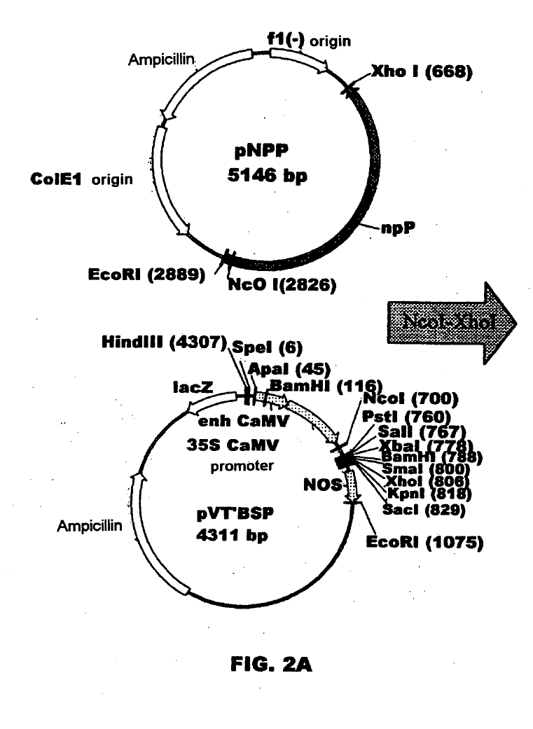

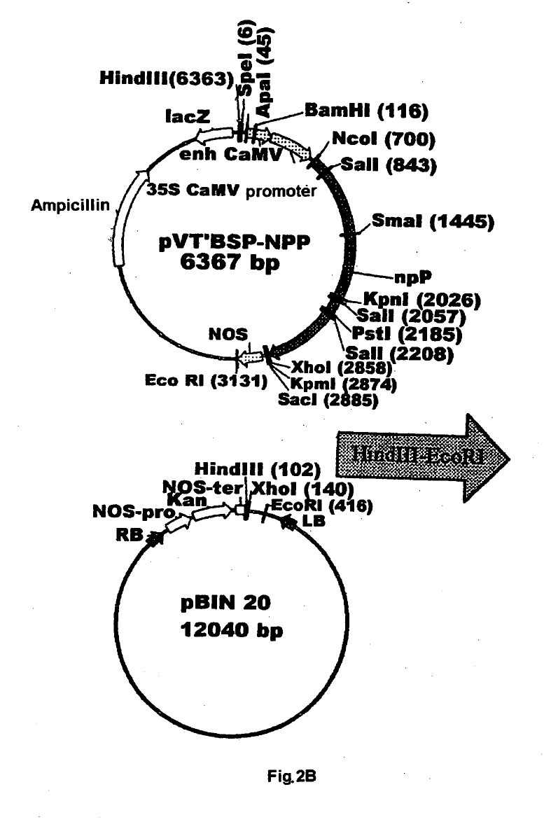

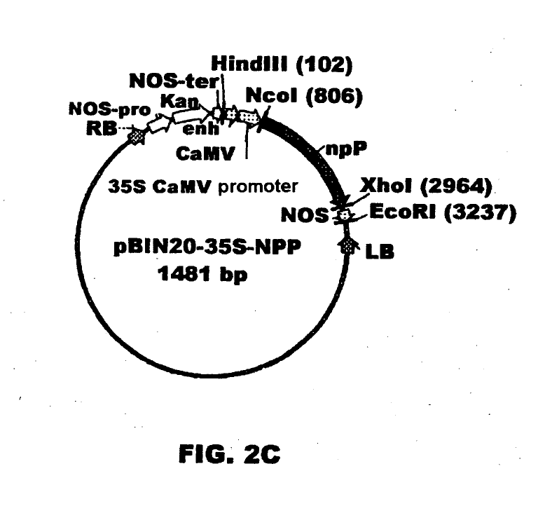

To be able to transfer this construction to the genome of the plants via Agrobacterium tumefaciens, it is necessary for it to be cloned previously in a binary plasmid. For this, p35SNPPNOS was digested successively with the enzymes HindIII, T4 DNA polymerase and XbaI and was cloned within the binary plasmid pBIN20 (Hennegan, K. P., Danna, K. J. “pBIN20: An improved binary vector for Agrobacterium-mediated transformation” Plant Mol. Biol. Rep. 16, 129-131) which had previously been digested successively with the enzymes HindIII, T4 DNA polymerase and XbaI. The plasmid thus obtained was designated with the name of pBIN20-35S-NPP (FIG. 2). After amplification in E. coli (XL1 Blue), pBIN20-35S-NPP was introduced into Agrobacterium tumefaciens, which was used for transforming species such as tomato, tobacco, potato etc. (Horsch, R. B., Fry, J. E., Hoffmann, N. L., Eichholtz, D., Rogers, S. G., Fraley, R. T. (1985) “A simple and general method for transferring genes into plants” Science 277, 1229-1231. The strain of Agrobacterium tumefaciens was deposited in the Spanish Type Culture Collection on 16 May 2003, located in the Research Building of the University of Valencia, Campus of Burjasot, Burjasot 46100 (Valencia, Spain) with the deposition number CECT 5799.

Preparation of Devices (Assay Kits) for Determination of Nucleoside Diphosphate Sugars and Sulphonucleotides

The kits designed for the determination of nucleotides such as nucleoside diphosphate sugars and sulphonucleotides are based on the action of the product with NPPase activity on phosphodiester and phosphosulphate bonds of small molecules which, after being hydrolysed, give rise to other molecules that are easy to detect and quantify.

The two most suitable strategies for making these kits start from the hydrolysis of the nucleoside diphosphate sugar by the enzyme according to the present invention, i.e. NPPase, producing equimolar quantities of sugar-1-phosphate and of the corresponding nucleoside monophosphate. From here on, consideration can be given to determination of the quantity of nucleotide initially present in the sample based on determination of the quantity of sugar-1-phosphate and nucleoside monophosphate produced, as specified below:
In the case when the sugar-1-phosphate is glucose-1-P (G1P), the said compound is submitted to the action of the enzyme phosphoglucomutase yielding glucose-6-phosphate, which in its turn can be made to undergo a coupling reaction with NAD⁺ by action of the enzyme glucose-6-phosphate dehydrogenase, obtaining 6-phosphogluconate and NADH, which is easily determined.
In the case when the sugar-1-phosphate is not G1P, its determination and that of the nucleoside monophosphate take place by colorimetric determination of the ortho phosphate (Pi) produced after the hydrolysis of these compounds with alkaline phosphatase. Alternatively, as the coupling enzyme it is possible to use 5′-nucleotidase, which will hydrolyse the nucleoside monophosphate to equimolar quantities of the corresponding nucleoside and Pi. The Pi released in either of the two cases is easily quantifiable by known colorimetric methods.

The strategy for determination of levels of sulphonucleotides such as APS is based on the hydrolysis of these nucleotides and consequent production of equimolar quantities of sulphate, which can be determined by turbidimetry or by nephelometry (Sörbo, B. (1987) “Sulfate: turbidimetric and nephelometric methods” Methods Enzymol. 143, 3-6).

Production of Polyclonal Antibodies Specific to Plant NPPase

Two milligrams of purified NPPase were separated in SDS-PAGE. After being eluted, it was mixed with complete Freund's adjuvant (at 50/50 ratio) and was then aliquoted in three equal fractions, each of which was injected into a rabbit at intervals of two weeks. Approximately three months after the first injection, the blood serum of the rabbit was extracted, which contains the polyclonal antibodies specific to AGPPase.

Identification of the Product by Western Blotting

Samples of proteins from wild plants and transgenic plants that overexpress the gene that codes for NPPase of rice were separated by SDS-PAGE. Then they were transferred to nitrocellulose membranes and the NPPase was detected using the specific anti-NPPase antibody according to the methodology described in the literature (Towbin, H., Staehelin, T., Gordon, J. (1979) “Electrophoretic transfer of proteins from polyacrylamide gels to nitrocellulose sheets: procedures and some applications” Proc. Natl. Acad. Sci. USA 76, 4350-4354.

### EXAMPLES OF APPLICATION OF THE INVENTION

Examples are presented below which describe in detail the method of production and purification of the soluble NPPase of young barley leaves. The same method, with slight changes appropriate to each case, can be applied to any other species. Other examples describe the use of NPPase for the production of assay kits for determination of nucleotide sugars and sulphonucleotides. Another example shows the production of a complete cDNA that codes for soluble NPPase.

### Example 1

### Extraction and Purification of the Soluble NPPase Obtained from Barley Leaves

All the steps were carried out at 4° C., unless indicated otherwise. The plant tissue (900 g) was homogenized with 2900 mL of extraction buffer (Mes 50 mM pH 6, EDTA 1 mM, DTT 2 mM) using a Waring blender. The homogenate was filtered through four layers of Miracloth, centrifuged at 100 000 g for 30 minutes and the supernatant was adjusted to 50% of ammonium sulphate. The precipitate obtained after 30 minutes of centrifugation at 30 000 g (20° C.) was resuspended in 2900 mL of Mes 50 mM pH 4.2, then heated on a water bath at 62° C. for 20 minutes, cooled in ice, and centrifuged at 30 000 g for 20 minutes. The proteins of the supernatant were precipitated using 50% ammonium sulphate, and resuspended in 5.7 mL of Mes 50 mM pH 6. Then the sample was submitted to gel filtration in a Superdex 200 column (Pharmacia LKB Biotechnology, Uppsala, Sweden) pre-equilibrated with Mes pH 6 and NaCl 150 mM. The fractions with NPPase activity were combined, concentrated, and submitted to isoelectric focusing in a native gel, permitting purification of the protein to homogeneity (FIG. 3).

### Example 2

### Enzymatic Tests

Unless stated otherwise, all the enzymatic reactions took place at 37° C. The determinations of NPPase activities were performed using spectrophotometric determination of G1P in two steps described by Sowokinos (1981) (Sowokinos, 1981, Plant Physiol. 68, 924-929). The reaction mixture contained Hepes 50 mM pH 7, the specified quantity of ADPG and the protein extract in a total volume of 50 microlitres. All the assays were performed relative to an ADPG blank. After 20 minutes of incubation, the reaction was stopped by boiling in a dry bath for 2 minutes. The mixture was centrifuged at 20 000 g for 5 minutes and the supernatant was recovered. In the second step, G1P was determined spectrophotometrically in 300 microlitres of mixture containing Hepes 50 mM⁻pH 7, EDTA 1 mM, MgCl₂ 2 mM, KCl 15 mM, NAD⁺ 0.6 mM, one unit of phosphoglucomutase and one unit of glucose-6-phosphate dehydrogenase from Leuconostoc mesenteroides, and 30 microlitres of the supernatant resulting from step 1. After 20 minutes of incubation, the production of NADH was monitored at 340 nm using a Multiskan EX spectrophotometer (Labsystems). The amount of NADH produced by any protein extract in the absence of ADPG in step 1 was negligible.

The native molecular weight of the NPPase was determined by gel filtration by plotting the partition coefficient (Kav) against the logarithm of the molecular weight of the following protein standards: bovine thyroglobulin (670 kDa), bovine gamma-globulin (158 kDa), ovalbumin (45 kDa), myoglobin (17 kDa) and vitamin B-12 (1.3 kDa). The protein content was determined by Bradford's method using the reagent made by Bio-Rad and gamma-globulin as standard.

Table 1 presented below shows the purification of soluble NPPase starting from barley leaves. The unit (U) is defined as the quantity of enzyme that catalyses the production of 1 μmol of product per minute.

TABLE 1

| | | | | Specific | |
|---|---|---|---|---|---|
| | Total | Total | Total | activity | Purifi- |
| | volume | protein | activity | (mU/mg | cation |
| | (mL) | (mg) | (mU) | protein) | (factor) |
| Supernatant | 2900 | 24 286 | 1 195 000 | 50 | — |
| 100000 × g | | | | | |
| pH 4.2/ | 2900 | 778 | 1 067 000 | 1370 | 28 |
| 62° C. | | | | | |
| Superdex | 86 | 164 | 300 | 1820 | 37 |
| 200 | | | | | |
| Isoelectric | 14 | 0.6 | 21 870 | 36 455 | 740 |
| focusing | | | | | |

### Example 3

### Identification of the Product with Enzymatic Activity Obtained

The product with NPPase activity thus obtained complies with the following characteristics:
It catalyses the hydrolysis of ADPG producing equimolar quantities of G1P and AMP.
In addition to ADPG, the NPPase recognizes other small molecules that possess phosphodiester bonds, such as UDPG, GDP-glucose, bis-PNPP and others of similar structure. It also catalyses the hydrolysis of small molecules with phosphosulphate bonds, such as APS, releasing equimolar quantities of sulphate and AMP (Table 2—Vmax in % in relation to ADPG—and Table 3).
It does not hydrolyse molecules with phosphomonoester bonds such as G1P, G6P, AMP, 3-phosphoglycerate, and other similar molecules. Nor does it hydrolyse cyclic AMP or nucleic acids such as DNA and RNA, which are substrates of other phosphodiesterases described in the literature.
Its ionic requirements are reduced, therefore the NPPase can work in the absence of ions of magnesium, manganese, cobalt, and other divalent cations, which are essential effectors for the functioning of other phosphodiesterases described in the literature.
In contrast to the pyrophosphatases of nucleoside diphosphate sugars of bacteria and animals, NPPase hydrolyses bis-PNPP.
It is inhibited by phosphorylated molecules such as AMP, ADP, ATP, 3-phosphoglycerate, orthophosphate, inorganic pyrophosphate, and others with similar characteristics.
It is strongly inhibited by molybdate and arsenate.
It is resistant to ionic detergents such as SDS (sodium dodecylsulphate).
It is resistant to the action of a wide range of proteases, such as Proteinase K and Pronase (Boehringer).
Its activity is not affected by the action of typical inhibitors of phosphodiesterases such as β-mercaptoethanol, EDTA, reduced cysteine, ascorbate, and other reducing and chelating agents.
It is sensitive to slightly basic pH and is very stable at pH between 4 and 7.5. This characteristic is one that makes NPPase a completely different enzyme from the majority of the phosphodiesterases described in the literature, as the latter are stable and active at slightly basic pH values.
It withstands a temperature of 65° C. for 30 minutes, and can be characterized by the following data:
Apparent molecular weight measured by gel filtration, around 70 kDa and 270 kDa, from which it is deduced that it has a monomeric form of 70 kDa and another homopolymeric form.
K_{eq}′ of the reaction 110.
Increase in standard free energy (ΔG′) of −2.9 kcal/mol.
It is a glycoprotein, since it is retained by columns of concanavalin.
Apparent molecular weight of the protein purified in denaturing gels, around 70 kDa.
The amino acid sequences of barley NPPase obtained are:
   N-terminal end: SEQ ID NO: 1
   Internal sequences (obtained after partial hydrolysis of NPPase with trypsin): SEQ ID NO: 2, 3, 4, 5 and 6.
The amino acid sequences of rice NPPase obtained are:
   N-terminal end: SEQ ID NO: 7

Internal sequences (obtained after partial hydrolysis of NPPase with trypsin): SEQ ID NO: 8, 9, 10, 11, 12, 13, 14, 15, 16 and 17.

TABLE 2
Kinetic parameters and substrate specificity
of NPPase from barley leaves

| substrate | Km (mM) | Vmax |
|---|---|---|
| ADPG | 0.5 | 100 |
| ATP | 3.5 | 40 |
| ADP | 3.5 | 40 |
| APS | 0.5 | 160 |
| PAPS | — | — |
| PAP | n.q. | n.q. |
| ADPmannose | 0.4 | 30 |
| GDPmannose | 0.4 | 30 |
| CDPG | 2.8 | 114 |
| UDPG | 2.1 | 114 |
| ADPribose | 2.4 | 100 |
| NAD⁺ | 2.5 | 100 |
| NADP⁺ | n.q. | n.q. |
| bis-PNPP | 0.3 | 100 |
| PNPP | 0.5 | 100 |
| Hexose monophospates | n.q. | n.q. |
| Nucleotide monophosphates | n.q. | n.q. |

TABLE 3
Kinetic parameters and substrate specificity
of the NPPase from rice leaves

| | | Vmax | k_{cat}/Kₘ |
|---|---|---|---|
| Substrate | Kₘ (mM) | (μmol min⁻¹ (mg protein)⁻¹) | (M⁻¹ s⁻¹) |
| ADP-glucose | 0.60 | 183 | 3.50 × 10⁵ |
| ADP-ribose | 1.43 | 317 | 2.48 × 10⁵ |
| UDP-glucose | 1.47 | 242 | 1.93 × 10⁵ |
| CDP-glucose | 0.92 | 107 | 1.93 × 10⁵ |
| GDP-glucose | 0.93 | 119 | 1.48 × 10⁵ |
| GDP-mannose | 0.80 | 129 | 1.90 × 10⁵ |
| TDP-glucose | 0.60 | 106 | 2.06 × 10⁵ |
| bis-PNPP | 1.30 | 191 | 1.71 × 10⁵ |
| ATP | 2.06 | 356 | 2.03 × 10⁵ |
| ADP | 4.33 | 385 | 1.00 × 10⁵ |
| AMP | n.d. | n.d. | — |
| PPi | — | 176 | — |
| APS | 3.04 | 282 | 1.09 × 10⁵ |

### Example 4

### Obtaining a Complete cDNA that Codes for the Soluble NPPase from Rice and an Incomplete cDNA that Codes for the Soluble NPPase of Barley

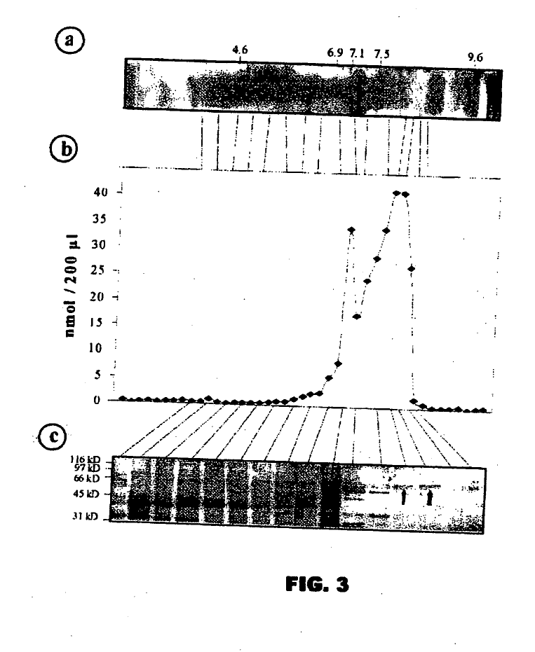

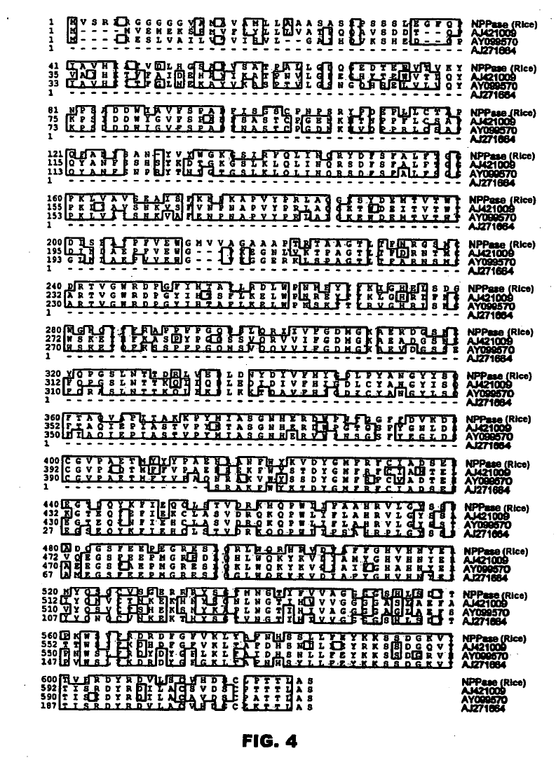

Knowledge of internal sequences of rice NPPase made it possible to design primers which, in the direction 5′-3′, are SEQ ID NO: 18 and 19. Using this primer, a cDNA was amplified by conventional methods of RT-PCR and was used as a probe for obtaining cDNA's from cDNA libraries from young leaves of rice and barley. As a result, a complete cDNA of rice NPPase was obtained and was inserted in a pSK Bluescript plasmid (Stratagene) which was amplified in the host bacterium XL1 Blue. The sequence of the complete cDNA of rice NPPase is SEQ ID NO: 20 and the amino acid deduced is SEQ ID NO: 21. An incomplete cDNA of barley NPPase was also obtained (SEQ ID NO: 22) and its deduced amino acid sequence is SEQ ID NO: 23. For this, the primer represented by SEQ ID NO: 24 was eliminated. After comparing with sequences available in databases, it was observed that the rice NPPase possesses 60% homology with PPD1 (access No. AJ421009), a nucleotide phosphatase/phosphodiesterase of L. luteus that hydrolyses nucleoside di- and tri-phosphates but is unable to hydrolyse nucleotide sugars (Olczak, M., Olczak, T. (2002) “Diphosphonucleotide phosphatase/phosphodiesterase from yellow lupin (Lupinus luteus L.) belongs to a novel group of specific metallophosphates”. FEBS Lett. 519, 159-163). Moreover, as shown in FIG. 4, cDNA's were sequenced for dicotyledonous plants such as Arabidopsis (access No. AY099570) and chickpea (access No. AJ271664) which code for unknown or possible proteins that show high homology with the rice NPPase.

### Example 5

### Products with NPPase Activity from Various Plants

The NPPase enzyme exhibits a very wide distribution among plants, so that the enzymatic product with NPPase activity can be obtained from any plant, especially from tissues of young plants such as leaves and roots. For example, the following Table 4 shows the specific activities (mU/mg protein) obtained in various monocotyledons and dicotyledons.

TABLE 4

| | Specific activity |
|---|---|
| | (mU/mg protein) |
| | (+ADPG) |
| Monocotyledons | |
| Leaf of barley (Hordeum vulgare) | 113.7 ± 3.5 |
| Leaf of wheat (Triticum aestivum) | 22.4 ± 2.5 |
| Dicotyledons | |
| Leaf of Arabidopsis thaliana (Wt) | 5.2 ± 0.6 |
| Leaf of pepper (Capsicum annuum) | 5.0 ± 0.6 |
| Leaf of tomato (Lycopersicon sculentum) | 5.6 ± 0.5 |
| Cell culture of sycamore (Acer pseudoplatanus) | 16.5 ± 7.2 |

### Example 6

### Preparation of Enzyme Kits for Determination of glucose-nucleoside diphosphates

For the determination of glucose-nucleoside diphosphates such as ADPG, UDPG, CDP-glucose, GDP-glucose and TMP-glucose, a kit is prepared containing the following elements:

a. NPPase

b. NAD

c. Phosphoglucomutase (PGM)

d. G6P dehydrogenase (G6PDH)

e. Buffer

Determination of the quantity of glucose-nucleoside diphosphate present in the test sample is based on spectrophotometric determination of the NADH produced according to the following coupled reaction:

$$( test sample ) {NAD}^{+} NADH$$

$$NDP \text{-} glucose -> NMP + G 1 P -> G 6 P -> 6 \text{-} phosphogluconate$$

$$\underline{NPPase} \underline{PGM} \underline{G 6 PDH}$$

The quantity of NDP-glucose in a test sample could be determined by preparing a cocktail with the composition (for 1 ml):

Test sample

1 U of NPPase

1 U of PGM

1 U of G6PDH

0.6 mM NAD

Buffer Mes or Hepes 50 mM pH 7

Water (make up to 1 ml)

Incubate at 37° C. for 20 minutes and observe the change in absorbance of the sample at 340 nm. A cocktail not containing NPPase can be used as a negative control.

### Example 7

### Preparation of Enzyme Kits for Determination of nucleoside diphosphates of Sugars Other than Glucose

Kits are prepared for determination of the following nucleoside diphosphate sugars:
ribose-nucleoside diphosphates (ADP-ribose, GDP-ribose, UDP-ribose, CDP-ribose or TDP-ribose)
mannose-nucleoside diphosphates (ADP-mannose, GDP-mannose, TDP-mannose, UDP-mannose or CDP-mannose)
galactose-nucleoside diphosphates (ADP-galactose, GDP-galactose, UDP-galactose or CDP-galactose)
glucuronic-nucleoside diphosphates (GDP-glucuronic, UDP-glucuronic, ADP-glucuronic, CDP-glucuronic or TDP-glucuronic)
fructose-nucleoside diphosphates (GDP-fructose, ADP-fructose, CDP-fructose, UDP-fructose, TDP-fructose)
galacturonic-nucleoside diphosphates (UDP-galacturonic, GDP-galacturonic, CDP-galacturonic, TDP-galacturonic or ADP-galacturonic)

The kit has the following elements:

a. NPPase

b. 5′-nucleotidase (or, alternatively, alkaline phosphatase)

c. buffer

Determination of the quantity of nucleoside diphosphate sugar present in the test sample is based on colorimetric determination of the orthophosphate released according to the following coupled enzymatic reaction:

$$( test sample )$$

$$NDP \text{-} sugar -> sugar \text{-} P + NMP -> base + Pi$$

$$\underline{NPPase} \underline{{5}^{′} \text{-} nucleotidase}$$

The Pi is determined according to any of the numerous colorimetric methods available in the bibliography and on the market.

The quantity of NDP-sugar in a test sample could be determined by preparing a cocktail (1 ml) consisting of:
Test sample
1 U of NPPase
1 U of 5′-nucleotidase (or, alternatively, 1 U of alkaline phosphatase)
Buffer Mes or Hepes 50 mM pH 7.5
Water (make up to 1 ml)

Incubate at 37° C. for 20 minutes and determine the production of Pi released according to conventional methods. A cocktail not containing NPPase can be used as negative control.

### Example 8

### Preparation of Enzyme Kits for Determination of PAPS and APS

The strategy for determination of levels of sulphonucleotides such as PAPS or APS is based on turbidimetric or nephelometric determination according to the following reaction:

$$\underline{NPPase}$$

$$PAPS -> PAP + sulfate$$

$$\underline{NPPase}$$

$$APS -> AMP + sulfate$$

The quantity of sulphonucleotide in a test sample could be determined by preparing a cocktail (1 ml) consisting of:
Test sample
1 U of NPPase
Buffer Mes or Hepes 50 mM pH 7.0
Water (make up to 1 ml)

Incubate at 37° C. for 20 minutes and determine the production of sulphate released according to conventional methods. A cocktail not containing NPPase can be used as negative control.

### Example 9

### Production of Transgenic Plants of Tobacco, Potato and Tomato that Overexpress NPPase

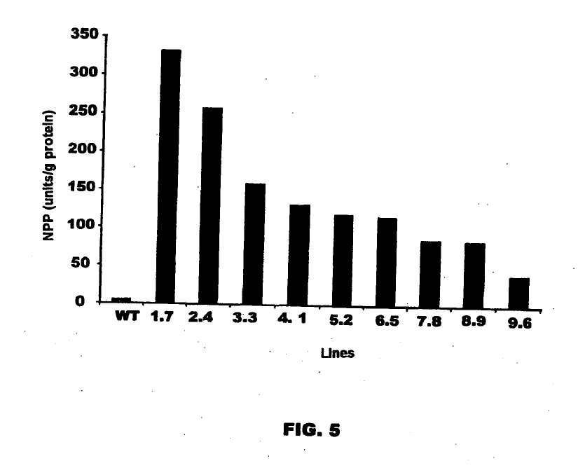

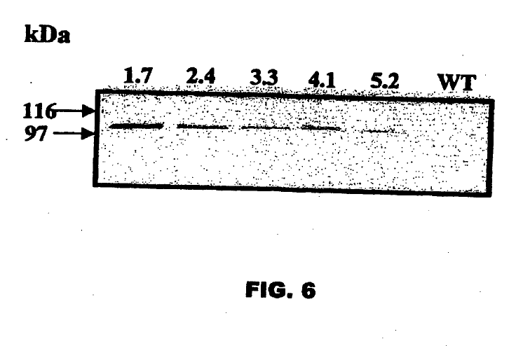

Using the strain of Agrobacterium tumefaciens CECT 5799, we obtained plants of tobacco (Nicotiana tabacum), potato (Solanum tuberosum) and tomato (Lycopersicon sculentum) with high NPPase activity in all the organs analysed (root, leaf, fruit and stalk) (FIG. 5). These plants accumulated large amounts of a protein that was recognized specifically by the polyclonal antibody obtained relative to rice NPPase (FIG. 6) and had the following characteristics:
1. Low content of starch and cell-wall carbohydrates (according to the measurement techniques based on commercial kits described in the literature (Frehner, M., Pozueta-Romero, J., Akazawa, T. (1990) “Enzyme sets of glycolysis, gluconeogenesis, and oxidative pentose phosphate pathway are not complete in nongreen highly purified amyloplasts of sycamore cell suspension cultures” Plant Physiol. 94, 538-544)).
2. High content of soluble sugars such as sucrose, glucose-6-phosphate, glucose and fructose.
3. Reduction of the levels of PAP accumulated in the tissues, imparting great resistance to high concentrations of sodium chloride in the growth substrate, relative to untransformed plants.
4. Resistance to high temperatures.

### DESCRIPTION OF THE DIAGRAMS

FIG. 1: Schematic diagram of pNPP

FIG. 2 A-C: Production of pBIN20-35S-NPP

FIG. 3: Isolation of the 70-kDa NPPase by isoelectric focusing (IEF) in a negative gel:
a) Corresponds to staining of the proteins after being separated by IEF, in relation to their isoelectric point (values shown at the top of the diagram). The cathode would be on the right and the anode on the left. A partially purified sample that contains 1 unit of AGPPase is applied to a plate of Ampholine PAG at a pH range 3.5-9.
b) Corresponds to a profile of NPPase activity. The NPPase activity of each fraction eluted from the IEF gel is measured.
c) Each fraction eluted from the IEF gel is submitted to SDS-PAGE and then staining with Coommassie-Blue. The fractions enriched with 70-kDa protein are indicated with an arrow and prove to be the most active enzymatically

FIG. 4: Comparison of amino acid sequences between the amino acid sequence deduced from the cDNA that codes for rice NPPase, and those deduced from the cDNA's that code for the PPD1 of Lupinus luteus (AJ421009) and unknown proteins of Arabidopsis (AY099570) and chickpea (AJ271664).

FIG. 5: Hydrolytic activities of ADPglucose in wild-type (WT) and 9 transgenic lines of potato that overexpress rice NPP.

FIG. 6: Western blot of 5 transgenic lines of potato that overexpress rice NPP. 50 micrograms of protein were loaded in each lane and were submitted to SDS-PAGE. After transfer to nitrocellulose filters, the NPP was immunodecorated specifically after using the polyclonal antibody specific to NPPs obtained in the rabbit.

1. An isolated enzyme product of plant origin designated NPPase, characterized in that its sequence contains at least one of the polypeptide fragments represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16 and SEQ ID NO: 17 and has phosphodiesterase activity.

2. The enzyme product designated NPPase, according to claim 1 characterized by having a amino acid sequence deduced from a cDNA selected among SEQ ID NO: 20 or SEQ ID NO: 22.

3. The enzyme product, according to claim 2 characterized in that it contains a sequence represented by SEQ ID NO: 21.

4. The enzyme product, according to claim 2 characterized in that it contains a sequence represented by SEQ ID NO: 23.

5. The enzyme product according to claim 1, characterized in that catalyses the hydrolysis of nucleotide sugars in equimolar conditions to sugar-phosphate and the corresponding nucleoside monophosphate, does not hydrolyse molecules with phosphomonoester bonds, is able to hydrolyse bis-PNPP, is inhibited by molybdate, arsenate and phosphorylated molecules, its activity is not affected by reducing and chelating agents that are inhibitors of phosphodiesterases, it is sensitive to slightly basic pH and is very stable at pH between 4 and 7.5, can be glycosylated, which makes it resistant to ionic detergents of the SDS type and to the action of proteases, and recognizes, in addition to nucleotide sugars, other small molecules that possess phosphodiester and phosphosulphate bonds.

6. The enzyme product as claimed claim 1 characterized in that it does not hydrolyse, among others, G1P, G6P, AMP, 3-phosphoglycerate, AMPc, nor nucleic acids.

7. The enzyme product as claimed in claim 1, characterized in that it does not require as effectors, among other divalent cations, magnesium, manganese or cobalt.

8. The enzyme product as claimed in claim 1, characterized in that it is inhibited by orthophosphate, inorganic pyrophosphate, and phosphate esters.

9. The enzyme product as claimed in claim 1, characterized in that its activity is not affected by, β-mercaptoethanol, EDTA, reduced cysteine or ascorbate.

10. The enzyme product as claimed in claim 1, characterized in that it is resistant to, Proteinase K or Pronase.

11. The enzyme product as claimed in claim 1, characterized in that it recognizes as a substrates, a compound selected from the group consisting of ADPG, UDPG, GDP-glucose, ADP-mannose, APS, PAPS or bis-PNPP.

12. The enzyme product as claimed in claim 1, characterized in that it is resistant to a temperature of 65° C. for 30 minutes, and in that it has an apparent molecular weight determined by gel filtration around 70 and 270 kDa for the monomeric and homopolymeric isoform respectively, and displays a K_{eq}′ of the reaction of 110, its G′ being −2.9 kcal/mol, and its Kₘ for ADPG being 0.5 mMolar.

13. The enzyme product as claimed in claim 1, characterized in that it is isolated from any plant species.

14. A method for obtaining an enzyme product of plant origin with nucleotide sugar pyrophosphatase/phospho-diesterase activity (NPPase) in its soluble isoform, having an amino acid sequence that contains at least one of the polypeptide fragments represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16 and SEQ ID NO: 17, comprising the steps of: extracting an extract from the material of plant origin using a buffer, said extract comprising a protein fraction, filtering the extract purifying the extract by successive centrifugations and precipitations, with adjustments both of the pH and of the ionic strength of the medium, preferably including heating of the protein above 60° C. and cooling in ice, and purification by gel filtration, isoelectric focusing, denaturing-gel electrophoresis, or other equivalent means of purification of proteins extracted from plant tissues.

15. The method as claimed in claim 14 comprising the following steps: (1) homogenization of the plant tissue with an extraction buffer, type Mes 50 mM pH 6, EDTA 1 mM, DTT 2 mM, (2) filtration, (3) ultracentrifugation at 100 000 g, (4) precipitation of the proteins of the supernatant in ammonium sulphate, (5) resuspension of the precipitate in buffer of pH 4.2, (6) heating for at least 15 minutes at a temperature between 60 and 65° C., followed by cooling in ice, (7) centrifugation at 30 000 g, (8) concentration of the protein of the supernatant by precipitation in ammonium sulphate and resuspension at pH 6, and (9) purification by gel-filtration chromatography, isoelectric focusing and denaturing-gel electrophoresis.

16. Primers represented by SEQ ID NO: 18, SEQ ID NO: 19 or SEQ ID NO: 24.

17. Use of a primers primer represented by SEQ ID NO: 18, SEQ ID NO: 19 or SEQ ID NO: 24 together with an mRNA from leaves of rice or barley for obtaining, by RT-PCR, a cDNA which, after being used as a probe on cDNA libraries of leaves of rice and barley, permits the isolation of cDNA's whose sequences are represented by SEQ ID NO: 20 and SEQ ID NO: 22, respectively.

18. cDNA represented by SEQ ID NO: 20 that codes for an enzyme product with NPPase activity.

19. Use of primers represented by SEQ ID NO: 18, SEQ ID NO: 19 or SEQ ID NO: 24 together with an mRNA from barley leaves for obtaining, by RT-PCR, a cDNA which, after being used as a probe on cDNA libraries of barley leaves, permits the isolation of cDNA whose sequence is represented by SEQ ID NO: 22.

20. cDNA represented by SEQ ID NO: 22 that codes for an enzyme product with NPPase activity.

21. Use of the enzyme product of claim 1 in the preparation of assay devices and/or compositions for application in the determination of nucleoside diphosphate sugars.

22. An assay device for the determination of nucleoside diphosphate sugars, characterized in that it includes the enzyme product of claim 3 in such a way that the determination is based on the sugar-1-phosphate released during the reaction catalysed by NPPase.

23. The assay device as claimed in claim 22, characterized in that the determination is based on the glucose-1-phosphate released, which is submitted to the enzyme phosphoglucomutase to produce glucose-6-phosphate, which in its turn is submitted to a coupled reaction with NAD⁺ and NADP⁺, by the action of the enzyme glucose-6-phosphate dehydrogenase to obtain 6-phosphogluconate and NADH or NADPH.

24. An assay device for the determination of nucleoside diphosphate sugars, characterized in that it includes the enzyme product of claim 1, in such a way that the determination is based on the nucleoside monophosphate produced during the reaction catalysed by NPPase.

25. The assay device as claimed in claim 24, characterized in that the determination is based on the nucleoside monophosphate, which is able to release orthophosphate, in addition to the corresponding base, by the action of an enzyme such as 5′-nucleotidase.

26. The assay device as claimed in claim 22, characterized in that the determination is based on the release of orthophosphate by the action of an enzyme such as alkaline phosphatase or 5′-nucleotidase from sugar-1-phosphate and monophosphate.

27. Use of the enzyme product of claim 1 in the preparation of an assay device and/or composition for application in the determination of the presence of 3′-phospho-adenosine 5′-phosphosulphate (PAPS) and adenosine 5′phosphosulphate (APS).

28. Use of the primer of claim 16 and cDNA represented by SEQ ID NO: 20 that codes for an enzyme product with NPPase activity in the production of transgenic plants that express or overexpress the cDNA that codes for NPPase.

29. A method for production of a transgenic plant that expresses or overexpresses the gene that codes for NPPase, characterized in that a transformation vector is used that contains a plasmid that includes the cDNA represented by SEQ ID NO: 20 of the gene of the NPPase.

30. A method for production of a transgenic plant that expresses or overexpresses the gene that codes for NPPase, characterized in that it uses a transformation vector that contains a plasmid that includes the cDNA represented by SEQ ID NO: 22 of the gene of the NPPase.

31. A method of production of transgenic plants as claimed in claim 29, characterized in that the transformation vector is Agrobacterium tumefaciens CECT 5799.

32. A transgenic plant obtainable by the method as claimed in claim 29, characterized in that it expresses or overexpresses the enzyme product of plant origin designated NPPase, characterized in that its sequence contains at least one of the polypeptide fragments represented by SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8; SEQ ID NO: 9; SEQ ID NO: 10; SEQ ID NO: 11; SEQ ID NO: 12; SEQ ID NO: 13; SEQ ID NO: 14; SEQ ID NO: 15; SEQ ID NO: 16 and SEQ ID NO: 17 and has phosphodiesterase activity and has a reduced content of starch and/or of cell-wall polysaccharides and is resistant to high temperature and to high salinity.

33. An assay device for the determination of sulphonucleotides, characterized in that it includes the enzyme product of claim 1 in such a way that the determination is based on the sulphate that is released.

34. The enzyme product according to claim 1 characterized in that it is inhibited by AMP, ADP, ATP, or 3-phosphoglycerate.

35. The enzyme product according to claim 1 where in the substrate is ADPG.

36. A method of production of transgenic plants as claimed in claim 30, characterized in that the transformation vector is Agrobacterium tumefaciens CECT 5799.
